(19) United States
(12) Patent Application Publication
(10) Pub. No.: US 2011/0015292 A1
(43) Pub. Date: Jan. 20, 2011

(54) PREPOLYMERS MADE FROM NATURAL OIL BASED POLYOLS

(75) Inventors: Bindushree Radhakrishnan, Lake Jackson, TX (US); William A. Koonce, Pearland, TX (US); Laura A. Grier, Brazoria, TX (US); Dwight D. Latham, Clute, TX (US)

Correspondence Address: The Dow Chemical Company, P.O. BOX 1967, 2040 Dow Center, Midland, MI, 48641, US

(73) Assignee: DOW GLOBAL TECHNOLOGIES INC., Midland, MI (US)

(21) Appl. No.: 12/933,284

(22) PCT Filed: Mar. 20, 2009
(86) PCT No.: PCT/US09/37816

§ 371 Date: Sep. 20, 2010

Related U.S. Application Data
(60) Provisional application No. 61/038,274, filed on Mar. 20, 2008.

(51) Int. Cl. C08G 18/00; C07C 269/02; C07F 7/08; C07D 303/02; C07C 309/00; C08J 9/00

(52) U.S. Cl. 521/170; 560/157; 528/85; 556/437; 549/512; 558/44

(57) ABSTRACT

A prepolymer and products made from the preopolymer is described. The prepolymer includes the reaction product of at least one isocyanate and at least one natural oil based polyol. The natural oil based polyol includes at least two natural oil moieties separated by a molecular structure having an average of at least about 19 ether groups between any 2 of the natural oil moieties or by a polyether molecular structure having an equivalent weight of at least about 480.

### CROSS-REFERENCE TO RELATED APPLICATIONS

This application claims benefit of U.S. Provisional Patent Application Ser. No. 61/038,274, Mar. 20, 2008, entitled “Polyether Natural Oil Polyols and Polymers Thereof” which is herein incorporated by reference.

### BACKGROUND

1. Field of the Invention

Embodiments of the present invention generally relate to polyurethane production; more specifically to polyol prepolymers useful in polyurethane production.

2. Description of the Related Art

Polyurethanes are used in a wide variety of applications, including cushioning foam, automotive body parts, wheels, casters and other cast and spray elastomers, structural foams, thermal insulating foam, coatings, adhesives and sealants. Aqueous polyurethane dispersions are used in a variety of films, coatings, adhesives and sealant applications. In addition, a great variety of hybrid polymeric materials exist which contain polyurethane or polyurea segments that are bonded into or blended with other polymer types. In order to form these various types of polyurethanes and hybrid materials, isocyanate-functional and isocyanate-reactive components are needed. In many cases, these components are prepared from simpler starting materials in order to obtain some application-specific advantage, such as a desirable viscosity, low volatile organic compound (VOC) contents, specific reactive groups, favorable component ratios, etc. This may be done by forming an isocyanate-terminated prepolymer through the reaction of an excess of a polyisocyanate with one or more isocyanate-reactive materials. However, the component ratios can be reversed to form an adduct having terminal hydroxyl or other isocyanate-reactive groups, if desired. The most common types of isocyanate-reactive materials are polyether polyols and polyester polyols. The polyether polyols are most typically a polymer of propylene oxide or a propylene oxide/ethylene oxide mixture.

These polyether and polyester polyols are often derived from oil, gas or coal feedstocks. These feedstocks are not renewable, and there is a desire to develop polyols that are derived from renewable resources. Various types of such polyols have been developed. However, these polyols may differ in structure, reactivity, polarity, compatibility and other physical and chemical characteristics from the commonly available polyether and polyester polyols, and therefore may not represent drop-in replacements for these materials in some applications.

### SUMMARY

In one embodiment of the invention a prepolymer and products made from the preopolymer is provided. The prepolymer includes the reaction product of at least one isocyanate and at least one natural oil based polyol. The natural oil based polyol includes at least two natural oil moieties separated by a molecular structure having an average of at least about 19 ether groups between any 2 of the natural oil moieties or by a polyether molecular structure having an equivalent weight of at least about 480.

In another embodiment, a method for producing a prepolymer is provided. The method includes reacting, at least, an isocyanate with a natural oil based polyol to form the prepolymer. The natural oil based polyol includes at least two natural oil moieties separated by a molecular structure having an average of at least about 19 ether groups between any 2 of the natural oil moieties or by a polyether molecular structure having an equivalent weight of at least about 480.

In another embodiment, a polyurethane product is provided. The polyurethane includes the reaction product of at least one isocyanate-reactive material and at least one prepolymer. The prepolymer includes the reaction product of at least one isocyanate and at least one natural oil based polyol. The natural oil based polyol includes at least two natural oil moieties separated by a molecular structure having an average of at least about 19 ether groups between any 2 of the natural oil moieties or by a polyether molecular structure having an equivalent weight of at least about 480.

In another embodiment, a method for producing a polyurethane product is provided. The method includes reacting at least one isocyanate-reactive material with at least one prepolymer. The prepolymer includes the reaction product of at least one isocyanate and at least one natural oil based polyol. The natural oil based polyol includes at least two natural oil moieties separated by a molecular structure having an average of at least about 19 ether groups between any 2 of the natural oil moieties or by a polyether molecular structure having an equivalent weight of at least about 480.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only exemplary embodiments of this invention and are therefore not to be considered limiting of its scope, for the invention may admit to other equally effective embodiments.

FIG. 1 is a plot of viscosity versus temperature of various prepolymers, including a comparison example and various embodiments of the invention.

FIG. 2 is a plot of the foaming profiles (height versus time) of various prepolymers, including comparison examples and various embodiments of the invention.

FIG. 3 is a plot of stress-strain behavior of various moisture cured films, including a comparison example and various embodiments of the invention.

FIG. 4 is a plot of stress-strain behavior of various moisture cured films after water exposure, including a comparison example and various embodiments of the invention.

### DETAILED DESCRIPTION

Embodiments of the invention provide for prepolymers and polyurethane products made from the prepolymers. The prepolymers may have at least one urethane group, and may at least be the reaction product of at least one isocyanate and at least one isocyanate-reactive material. The isocyanate-reactive material may comprise at least one polyether natural oil based polyol (PNOBP). The PNOBP may include at least two natural oil moieties separated by a molecular structure having an average of at least about 19 ether groups between any 2 of the natural oil moieties or by a polyether molecular structure having an equivalent weight of at least about 480. The prepolymer is useful to make a variety of polymeric materials, including polyurethanes, polyureas, polyesters, UV-curable materials, various hybrid polymers, and the like.

The prepolymer may be characterized as having an average of at least one urethane group per molecule, and typically may contain more than one urethane group per molecule. The number of urethane groups may be determined in each instance by the functionality of the isocyanate-reactive material(s) (i.e., number of isocyanate-reactive groups per molecule), the functionality of the isocyanate compound and the stoichiometric ratio of isocyanate-reactive material(s) and isocyanate compounds that are used to prepare the prepolymer. The prepolymer may be further characterized as having reactive functional groups such as isocyanate, hydroxyl, carboxylic acid, carboxylic acid anhydride, epoxide, amino, silane or ethylenic unsaturation.

The prepolymer may be a liquid at room temperature (˜22° C.) or if a solid, one that has a melting temperature of no greater than 80° C., especially no greater than 50° C.

Suitable isocyanates for use in preparing the prepolyomer include a wide variety of organic mono- and polyisocyanates. Suitable monoisocyanates include benzyl isocyanate, toluene isocyanate, phenyl isocyanate and alkyl isocyanates in which the alkyl group contains from 1 to 12 carbon atoms. Suitable polyisocyanates include aromatic, cycloaliphatic and aliphatic isocyanates. Exemplary polyisocyanates include m-phenylene diisocyanate, tolylene-2-4-diisocyanate, tolylene-2-6-diisocyanate, isophorone diisocyanate, 1,3- and/or 1,4-bis(isocyanatomethyl)cyclohexane (including cis- or trans-isomers of either), hexamethylene-1,6-diisocyanate, tetramethylene-1,4-diisocyanate, cyclohexane-1,4-diisocyanate, hexahydrotolylene diisocyanate, methylene bis(cyclohexaneisocyanate) (H₁₂MDI), naphthylene-1,5-diisocyanate, methoxyphenyl-2,4-diisocyanate, diphenylmethane-4,4′-diisocyanate, 4,4′-biphenylene diisocyanate, 3,3′-dimethoxy-4,4′-biphenyl diisocyanate, 3,3′-dimethyl-4-4′-biphenyl diisocyanate, 3,3′-dimethyldiphenyl methane-4,4′-diisocyanate, 4,4′,4″-triphenyl methane triisocyanate, a polymethylene polyphenylisocyanate (PMDI), tolylene-2,4,6-triisocyanate and 4,4′-dimethyldiphenylmethane-2,2′,5,5′-tetraisocyanate. In some embodiments, the polyisocyanate is diphenylmethane-4,4′-diisocyanate, diphenylmethane-2,4′-diisocyanate, PMDI, tolylene-2,4-diisocyanate, tolylene-2,6-diisocyanate or mixtures thereof. Diphenylmethane-4,4′-diisocyanate, diphenylmethane-2,4′-diisocyanate and mixtures thereof are generically referred to as MDI, and all may be used. Tolylene-2,4-diisocyanate, tolylene-2,6-diisocyanate and mixtures thereof are generically referred to as TDI, and all may be used.

Derivatives of any of the foregoing polyisocyanate groups that contain biuret, urea, carbodiimide, allophonate and/or isocyanurate groups may also be used. These derivatives often have increased isocyanate functionalities and are desirably used when a more highly crosslinked product is desired.

The isocyanate-reactive material includes least one polyether natural oil based polyol (PNOBP). The polyether PNOBP may be made by reacting an initiator with a natural oil or derivative thereof, such as a natural oil based monomer such as is described in WO2004096882 which is hereby incorporated herein by reference. The initiator may have at least one active hydrogen, which is reacted with the natural oil based monomer, and has sufficient ether groups to render it more compatible or miscible with water, conventional polyether polyols or a combination thereof or to improve processibility or physical properties. Such initiators are referred to herein as polyether initiators, and includes amine tipped polyethers. In one embodiment, a PNOBP is made with an initiator or combination of initiators having an average equivalent weight of between about 480 and about 3000 per active hydrogen group. All individual values and subranges between about 480 and about 3000 per active hydrogen group are included herein and disclosed herein; for example, the average equivalent weight can be from a lower limit of about 480, 500, 550, 600, 650, 700, 800, 900, 1000, 1200, or 1300 to an upper limit of about 1500, 1750, 2000, 2250, 2500, 2750, or 3000 per active hydrogen group.

Thus, at least two of the natural oil based monomers are separated by a molecular structure having an average molecular weight of between about 1250 Daltons and about 6000 Daltons. All individual values and subranges between about 1250 Daltons and about 6000 Daltons are included herein and disclosed herein; for example, the average molecular weight can be from a lower limit of about 1250, 1500, 1750, 2000, 2250, 2500, 2750, 3000, or Daltons to an upper limit of about 3000, 3500, 4000, 4500, 5000, 5500, or 6000 Daltons.

In one embodiment, these characteristics are achieved using a single initiator, optionally with those impurities present in commercial products. In an alternative embodiment, the characteristics are achieved using combinations (referred to hereinafter as blends, mixtures or admixtures) of initiators in making the PNOBP and/or in combinations of natural oil based monomers. In either combination, at least about 10, at least about 15, at least about 20, preferably at least about 25, or at least about 30 weight percent (mass fraction) of the initiator used has an equivalent weight of at least about 480. When more than one initiator is used, the PNOBPs may be prepared separately with the resulting products combined in physical blends, used together in the same reaction to form insitu combinations, or a combination thereof.

The ether groups may be in poly(alkylene oxide) chains, such as in poly(propylene oxide) or poly(ethylene oxide) or a combination thereof. In one embodiment, the ether groups may be in a diblock structure of poly(propylene oxide) capped with poly(ethylene oxide).

The active hydrogen group is optionally any active hydrogen group that is sufficiently reactive to react with the natural oil or derivatives thereof under reaction conditions, and each active hydrogen group may be independently a hydroxyl or amine group. For example, the active hydrogen group may be a hydroxyl group. In one embodiment the hydroxyl group may be a primary hydroxyl group. In the case of amine groups, primary and secondary amines may be used. Of the active hydrogen groups, at least about 50, 60, 70, 80, 85, 90, or up to 100 mole percent of these groups are primary hydroxyl groups or amine groups. In one embodiment, these amounts of primary hydroxyl groups in the initiator may also be the amounts of primary hydroxyl group in the PNOBP produced.

Thus the initiators are may be depicted by Formula 1:

R((OCH₂CHY)_{b}—XH)ₚ

where Y is a H, CH₃ or higher alkyl group (preferably C1 to C16, preferably C1 to C8, or preferably C1 to C4) or mixture thereof; X is an active hydrogen group, independently preferably O, N, or NH, or preferably O; p is 1 to 8, preferably 2 to 8; b is sufficient to result in an equivalent weight per active hydrogen group of at least about 480, preferably at least about 7 to a most about 70. The number of ether units in an arm of the polyether initiator, b, may be at least about 9, or at least about 12, when the equivalent weight is at least about 480, but at least about 13, at least about 14, or at least about 15, when the equivalent weight is less than about 480; and regardless of equivalent weight, b is independently may be at most about 70, at most about 55, or at most about 45, such on average, the equivalent weight of the compound of Formula 1 is at least about 480, or on average each active hydrogen is separated from each other active hydrogen by an average of 19 ether groups (—OCH₂CHY—), preferably both. In the formula, each X is optionally the same or different. The initiator, therefore, encompasses polyols, polyamines and aminoalcohols. R generally represents a linear, cyclic chain or combination thereof of alkane (C—C), alkene (C═C), ether (C—O—C) linkages or combinations thereof. R may have at least about 1, at least about 2, or at least about 3, and independently preferably has at most about 36, at most about 24, or at most about 12 carbon atoms. The carbon atoms within the aforementioned chain are optionally substituted with a methyl or ethyl group. It should be noted that the value of each b in a polyether initiator optionally is the same or varies from one OCH₂CHY)_{b}—XH chain or “arm” of the polyether initiator to another. Furthermore, those skilled in the art will recognize that there will be variations in the numbers of alkylene oxide molecules added to a molecule in a reaction, thus in the value of b with in a molecule of polyether initiator and among molecules prepared simultaneously. To allow for variations, values of b previously listed are understood to be the average b over all chains of the polyether initiator or combination thereof.

The R group is optionally exemplified by polyol initiators for polyethers that include neopentylglycol; 1,2-propylene glycol; trimethylolpropane; pentaerythritol; sorbitol; sucrose; glycerol; alkanediols such as 1,6-hexanediol; 2,5-hexanediol; 1,4-butanediol; 1,4-cyclohexane diol; ethylene glycol; diethylene glycol; triethylene glycol; 9(1)-hydroxymethyloctadecanol, 1,4-bishydroxymethylcyclohexane; 8,8-bis(hydroxymethyl)tricyclo[5,2,1,02,6]decene; Dimerol alcohol (36 carbon diol available from Henkel Corporation); hydrogenated bisphenol; 9,9(10,10)-bishydroxymethyloctadecanol; 1,2,6-hexanetriol; any of the aforementioned where at least one of the alcohol or amine groups present therein has been reacted with ethylene oxide, propylene oxide or mixture thereof; and combination thereof.

Exemplary polyamines that can form the R group of Formula 1 include ethylene diamine; neopentyldiamine, 1,6-diaminohexane; bisaminomethyltricyclodecane; bisaminocyclohexane; diethylene triamine; bis-3-aminopropyl methylamine; and triethylene tetramine. Exemplary aminoalcohols include ethanolamine, diethanolamine, and triethanolamine. Other compounds that are optionally used include polyols, polyamines or aminoalcohols described in U.S. Pat. Nos. 4,216,344; 4,243,818 and 4,348,543 and British Pat. No. 1,043,507.

Preferably, the initiator that forms R may be selected from the group consisting of neopentylglycol; trimethylolpropane; pentaerythritol; sorbitol; sucrose; glycerol; 1,2-propylene glycol; 1,6-hexanediol; 2,5-hexanediol; 1,6-hexanediol; 1,4-cyclohexane diol; 1,4-butanediol; ethylene glycol; diethylene glycol; triethylene glycol; bis-3-aminopropyl methylamine; ethylene diamine; diethylene triamine; 9(1)-hydroxymethyloctadecanol; 1,4-bishydroxymethylcyclohexane; 8,8-bis(hydroxymethyl)tricyclo [5,2,1,02,6]decene; Dimerol alcohol; hydrogenated bisphenol; 9,9(10,10)-bishydroxymethyloctadecanol; 1,2,6-hexanetriol; any of the aforementioned where at least one of the alcohol or amine groups present therein has been reacted with ethylene oxide, propylene oxide or mixture thereof; and combination thereof.

Then, to form the polyether initiator, the active hydrogen groups may be reacted with at least one alkylene oxide, such ethylene oxide or propylene oxide or a combination thereof; or a block of propylene oxide followed by a block of ethylene oxide, to form a polyether polyol by means within the skill in the art. The polyether polyol is may be used as a polyol for reaction with at least one natural oil or derivative thereof or with at least one natural oil based monomer. Alternatively the polyol is reacted by means within the skill in the art to convert one or more hydroxyl groups to alternative active hydrogen groups, such as is propylene oxide.

The polyether initiator is reacted with at least one natural oil or derivative thereof, such as at least one natural oil based monomer such as is described in WO2004096882. The natural oil or derivative thereof is optionally any natural oil or derivative of a natural oil reactive with at least one active hydrogen group on a polyether initiator according to the practice of the embodiments of the invention. Preferably the natural oil or derivative thereof has at least one acid, anhydride, acid chloride, or ester group reactive with at least one active hydrogen group on a polyether initiator to form at least one ester or amide. The natural oils or derivatives thereof are exemplified by natural oil based monomers herein, but the exemplification is not intended to limit the embodiments of the invention to the natural oil based monomers.

The natural oil based monomer or other fatty acid or derivative thereof is optionally formed from of any animal fat or vegetable oil that is comprised of triglycerides that upon saponification with a base such as aqueous sodium hydroxide yields a fatty acid and glycerol, where at least a portion of the fatty acids are preferably unsaturated fatty acids (that is, contain at least one carbon double bond). Preferred vegetable oils are those that yield at least about 70 percent unsaturated fatty acids by weight. More preferably, the vegetable oil yields at least about 85 percent, at least 87 percent, or at least about 90 percent by weight unsaturated fatty acids. It is understood that specific fatty acids derivable from a vegetable oil, animal fat or any other source are optionally used. That is to say, for example, palmitoleic, oleic, linoleic, linolenic and arachidonic fatty acid alkyl esters are optionally used to form the natural oil based monomer directly. Examples of suitable vegetable oils include, for example, those from castor, soybean, olive, peanut, rapeseed, corn, sesame, cotton, canola, safflower, linseed, palm, grapeseed, black caraway, pumpkin kernel, borage seed, wood germ, apricot kernel, pistachio, almond, macadamia nut, avocado, sea buckthorn, hemp, hazelnut, evening primrose, wild rose, thistle, walnut, sunflower, jatropha seed oils, or a combination thereof. Additionally, oils obtained from organisms such as algae may also be used. Examples of animal products include lard, beef tallow, fish oils and mixtures thereof. A combination of vegetable and animal based oils/fats may also be used. It is understood that the vegetable oil is optionally obtained from a genetically modified organism, such as genetically modified soybean, sunflower or canola.

Unsaturated fatty acid alkyl esters may then be formed, by any suitable process such as those known in the art, into preferred natural oil based monomers. For example, the hydroxymethyl group is optionally introduced by a hydroformylation process using a cobalt or rhodium catalyst followed by the hydrogenation of the formyl group to obtain the hydroxymethyl group by catalytic or by chemical reduction. Procedures to form the hydroxymethyl esters are described in U.S. Pat. Nos. 4,216,343; 4,216,344; 4,304,945 and 4,229,562 and in particular 4,083,816. Other known processes to form hydroxymethyl esters from fatty acids may also be used such as described by U.S. Pat. Nos. 2,3324,849 and 3,787,459.

In forming the natural oil based monomers, fatty acid alkyl esters are optionally completely formylated or only partially formylated. That is to say, the fatty acid alkyl esters of the particular vegetable oil optionally have some remaining unsaturated (C═C) bonds. Preferably, however, the amount of unsaturated bonds remaining after formylation is as described in WO2004096744, incorporated herein by reference. After the fatty acid alkyl esters are formylated they may be hydrogenated, such that there is desirably essentially no remaining unsaturated bonds (that is, trace amounts at most and preferably no detectable amounts of unsaturation).

At least one natural oil or derivative thereof and at least one polyether initiator are reacted together by any suitable means such as those known in the art to form at least one PNOBP. For example, the method is taught in WO200496882 and WO2004096883, which are incorporated herein by reference. The natural oil moiety may optionally be reacted with the initiator before or after functionalization, that is, formation or introduction of hydroxyl groups or their precursors to the fatty acid moieties.

In an embodiment, a functionalized natural oil moiety is formed, and then is reacted with a polyether initiator by any means within the skill in the art, for instance, transesterification, wherein an ester linkage is formed by reaction of a polyether initiator with the methyl ester of a functionalized fatty acid or, alternatively by esterification of an acid, chloride or anhydride form of the natural oil or derivative. The natural oil moiety of this embodiment is optionally functionalized by any means within the skill in the art, for example by epoxidation (and ring opening), amination, reacting with such compounds as maleic anhydride or perchloric acid, air oxidation, ozonolysis, hydroformylation, reaction with water such as blown oils where moist air in the presence of a catalyst preferably by epoxidation or hydroformylation.

In an alternative embodiment, the natural oil based monomer may be an unsaturated fatty acid unit in the acid form or in the methyl ester form. This monomer unit is optionally reacted with the polyether initiator (or combination thereof) using the same chemistry used for reaction with the functionalized natural oil based monomer. After this natural oil based monomer is reacted with the polyether initiator; it is then functionalized by any reaction within the skill in the art, such as those listed for functionalization before reaction with the polyether initiator. The functional group is directly useful for the formation of polyurethanes, or optionally undergoes further chemical reaction to form a useful functional group, such as the ring opening of an epoxy functional group to form the a NOP useful for such purposes.

The resulting PNOBP, comprises at least two natural oil moieties separated by a molecular structure having at least about 19 ether groups or having an equivalent weight of at least about 480, preferably both. When the polyether initiator has more than 2 active hydrogen groups reactive with the natural oil or derivative thereof, each natural oil moiety is separated from another by an average of at least about 19 ether groups or a structure of molecular weight of at least about 480, preferably both.

Thus, the PNOBP's are represented by Formula 2:

R(OCH₂CHY)_{b}XQ)ₚ,

wherein R, X, b, and p are as defined for Formula 1 and each Q independently represents at least one natural oil moiety. The Q's of a molecule are optionally the same or different. Q advantageously has the structure of at least one natural oil or, of one or more fatty acids or derivatives thereof, or at least one hydroxy functional fatty acid or derivative thereof, or at least one hydroxymethyl methyl fatty acid or derivative thereof. Q may also represent a series of fatty acid derivatives, most preferably oligomerized by esterification or transesterification of at least one hydroxyl group or ester group, preferably the hydroxyl of a hydroxymethyl group on each fatty acid derivative with the acid or ester (preferably methyl ester) of another fatty acid derivative molecule or molecular portion. Preferably at least about an average of 0.5, 0.8, or 1 fatty acid are oligomerized to form each natural oil moiety, Q. The number of fatty acid or fatty acid derivatives in each Q is preferably at most about 8, at most about 5, or at most about 3.

The isocyanate-reactive material may in addition to PNOBP include one or more other materials that have isocyanate-reactive groups, including materials having one or more hydroxyl, primary amine, secondary amine or epoxide groups. These other isocyanate-reactive materials may be of various types. For example, other isocyanate-reactive materials having an equivalent of 400 or greater, especially from about 400 to about 8,000, or about 500 to about 3,000 or from about 600 to about 2,000 can be used. Examples of such higher equivalent weight materials include polyether polyols, polyester polyols, and aminated polyethers. They typically will have a functionality (isocyanate-reactive groups/molecule) of about 1 to about 8, especially from about 1.8 to about 3.5. The polyethers of interest include homopolymers of propylene oxide, ethylene oxide or tetrahydrofuran, for example, and random and/or block copolymer of propylene oxide and ethylene oxide. Polyesters of interest include polylactones and butanediol/adipate polyesters.

The isocyanate-reactive material may also include a chain extender. For the purposes of the various embodiments of the invention, a chain extender means a material having two isocyanate-reactive groups per molecule and an equivalent weight per isocyanate-reactive group of less than 400, preferably less than 200 and especially from 31 to 125. The isocyanate reactive groups are preferably hydroxyl, primary aliphatic or aromatic amine or secondary aliphatic or aromatic amine groups. Representative chain extenders include amines ethylene glycol, diethylene glycol, 1,2-propylene glycol, dipropylene glycol, tripropylene glycol, cyclohexane dimethanol, ethylene diamine, phenylene diamine, bis(3-chloro-4-aminophenyl)methane and 2,4-diamino-3,5-diethyl toluene.

The polyol component may contain one or more crosslinkers in addition to the high equivalent weight polyols described above. For purposes of the embodiments of the invention “crosslinkers” are materials having three or more isocyanate-reactive groups per molecule and an equivalent weight per isocyanate-reactive group of less than 400. Crosslinkers may contain from 3 to 8, especially from 3 to 4 hydroxyl, primary amine or secondary amine groups per molecule and have an equivalent weight of from 30 to about 200, especially from 50 to 125. Examples of suitable crosslinkers include diethanol amine, monoethanol amine, triethanol amine, mono- di- or tri(isopropanol) amine, glycerine, trimethylol propane, pentaerythritol, and the like.

The PNOBP may constitute at least 10%, at least 25%, at least at least 35%, at least 50%, or at least 65% of the total weight of the isocyanate-reactive materials used to make the prepolymer. The PNOBP may constitute 75% or more, 85% or more, 90% or more, 95% or more or even 100% of the total weight of the isocyanate-reactive material. For example, the PNOBP(s) may constitute from 20 to 65%, from 35 to 65%, from 65 to 100% or from 80 to 100% of the total weight of isocyanate-reactive materials.

The prepolymer may have a variety of types of functional groups, depending on the particular starting materials used and their relative proportions. In one embodiment the functional group is an isocyanate group. The prepolymer may have isocyanate-reactive functional groups, such as hydroxyl or primary or secondary amine groups. The prepolymer may have other types of functional groups such as epoxide, carboxylic acid, carboxylic acid anhydride or similar groups.

Isocyanate-Functional Prepolymers

Isocyanate-functional prepolymers are of particular interest, as they form starting materials that are useful to prepare a wide variety of polyurethane and polyurethane-urea products. These materials are characterized by having an average of at least one, preferably an average of at least 1.8, isocyanate groups/molecule. These materials may contain from about 2.0, or from about 2.2, or from about 2.5, to about 6, to about 4, to about 3.5 or to about 3.0 isocyanate groups/molecule, on average.

A polyisocyanate is suitably used to prepare an isocyanate-functional prepolymer of the embodiments of the invention, although mixtures of mono- and polyisocyanates can be used. In order to produce a low molecular weight product that is liquid or low-temperature melting, the isocyanate(s) are used in an amount that provides greater than one equivalent of isocyanate groups per equivalent of isocyanate-reactive group present in the isocyanate-reactive mixture. It is preferred to provide at least 1.5, especially at least 1.8, even more preferably at least 2.0 equivalents of isocyanate groups/equivalent of isocyanate-reactive group.

When approximately one mole (such as from about 0.85 to about 1.15 or from about 0.95 to about 1.05 moles) of polyisocyanate compounds are used per equivalent of isocyanate-reactive groups, the isocyanate-reactive materials become “capped” with the polyisocyanate. The resulting prepolymer is a statistical distribution of products of various molecular weights, as well as some unreacted monomer. The average molecular weight closely approximates MW_{(isocyanate-reactive material)}+nMW_{(isocyanate)}, where n is the number of isocyanate-reactive groups per molecule of isocyanate-reactive material. In this case, relatively few unreacted isocyanate-reactive groups remain and there is little if any unreacted isocyanate compound. These prepolymers thus have the advantage of having low levels of volatile isocyanate compounds. Isocyanate-terminated prepolymers of this type are useful in a variety of applications, such as cast and spray elastomer applications, moisture-curable sealants and adhesives, two-part sealants and adhesives, aqueous polyurethane dispersions, and the like. In each instance, the prepolymer may be tailored in specific ways to adapt it for its particular usage.

For cast elastomer applications, aromatic polyisocyanates such as toluene diisocyanate, MDI, and PMDI, or mixtures thereof with an aliphatic polyisocyanate are preferred, except when high stability to light is needed. When good light stability is needed, aliphatic polyisocyanates such as H₁₂MDI, isophorone diisocyanate, 1,3- and/or 1,4-bis(isocyanatomethyl)cyclohexane and hexamethylene diisocyanate are preferable. The isocyanate-reactive material may contain only the PNOBP. If other isocyanate-reactive materials are used, they are preferably one or more high equivalent weight polyols such as a 400-6,000 equivalent weight polyether polyol, especially a poly(propylene oxide) or propylene oxide/ethylene oxide copolymer in which polymerized propylene oxide constitutes at least 80 and especially at least 90% of the total polyol weight. The polyether polyol may contain mainly secondary hydroxyl groups. This polyether polyol preferably has a functionality of from 1.8 to about 3, especially from about 2 to about 3. The additional high equivalent weight polyol(s) suitably constitute from about 20 to about 65% of the total weight of the isocyanate-reactive compounds used to make the prepolymer. It is also possible to include a small amount of chain extenders and/or crosslinkers in the isocyanate-reactive material. These typically will constitute no more than about 20%, especially up to about 10% of the total weight of the isocyanate-reactive materials. This prepolymer most suitably has an isocyanate equivalent weight of about 500 to about 8,000.

A similar prepolymer is suitable for one-part, moisture curable adhesive and sealant applications. Here, the PNOBP is used alone as the sole component of the isocyanate reactive material, or else is suitably used in conjunction with a high equivalent weight polyether polyol or polyester polyol and/or a chain extender. The high equivalent weight polyether polyol is preferably a poly(propylene oxide) or copolymer of poly(propylene oxide) and up to about 12% by weight ethylene oxide, having a functionality of up to about 3 and an equivalent weight of about 500 to about 1500.

Yet another application for these prepolymers as a binder for use in making rebounded foam cushion and for recycling rubber crumb into molded items or to make sport floor systems for indoor and/or outdoor use.

When more than one mole of polyisocyanate or polyisocyanate/monoisocyanate mixture is used per equivalent of isocyanate-reactive material, the resulting product generally is a mixture of an isocyanate-terminated prepolymer as described before and some amount of unreacted isocyanate compounds. Mixtures of this type are often referred to in the art as “quasi-prepolymers”. They may have an isocyanate content up to about 30% by weight, such as from 20 to 30% by weight. Quasi-prepolymers can be used in the same applications in which the above-described prepolymers are used. In addition, quasi-prepolymers of this type are particularly useful in making polyurethane elastomers in a so-called reaction injection molding (RIM), structural reaction injection molding (SRIM) or reinforced reaction injection molding (RRIM) process. The quasi-prepolymers are also particularly useful in making molded foam and microcellular elastomers. For all of these applications, the polyisocyanates may include TDI, MDI, carbodiimide-modified MDI (such as Isonate® 143L from Dow Chemical) and polymeric MDI.

The quasi-prepolymer may contain about 1 to about 60, preferably from about 5 to about 40%, by weight unreacted polyisocyanate compound. The PNOBP may be used as the sole isocyanate-reactive material, or it may be blended with one or more other high equivalent weight materials such as polyether polyols or another polyester polyol. For RIM, SRIM and RRIM applications, it may be desirable to include a chain extender and/or crosslinker in the isocyanate-reactive materials. Quasi-prepolymers of particular interest are made with a blend of the PNOBP and a crosslinker and/or chain extender. The amount of crosslinker and/or chain extender is such that the combined weight of the isocyanate compounds and crosslinker and/or chain extender, is from about 5 to about 80% of the total weight of the isocyanate compound and all isocyanate-reactive materials. This ratio of isocyanate compounds plus crosslinker and/or chain extender to total weight of reactants is sometimes referred to herein as the “hard segment content”. Hard segment contents of from 20 to 60%, especially from 25 to 45% are of particular interest in quasi-prepolymers for RIM, SRIM and RRIM applications.

When less than one mole of isocyanate compound is used per equivalent of isocyanate-reactive materials, the resulting product will tend to be a mixture of materials that include a significant number of species in which two or more molecules of the isocyanate-reactive material(s) are coupled together to form a higher molecular weight oligomer.

Isocyanate-terminated prepolymers of the embodiments of the invention can be tailored for specific applications by adjusting the ratios of components and in some instances by the selection of additional isocyanate-reactive materials. A first example of this is the control of hard segment content, primarily through the use of lower equivalent weight PNOBP materials and the use of chain extender and/or crosslinker materials in making the prepolymer. In general, the use of higher hard segment content prepolymer tends to form a harder, more crosslinked and less elastomeric polymer when cured. For example, elastomeric polyurethanes generally are made using a prepolymer having a hard segment content of 5 to 30% by weight, whereas structural elastomers (such as RIM, SRIM and RRIM) tend to employ prepolymers having a hard segment content of from 25 to 60%, especially from 25 to 45% by weight. For forming hard coatings for wood and metal, a prepolymer having a hard segment content of from 30 to 60% is often used.

Another example of this is an isocyanate-terminated prepolymer that is water-dispersible. Such prepolymers are useful in making aqueous polyurethane/urea dispersions, because the prepolymer is sufficiently water-dispersible that it can form stable droplets in an aqueous phase. This water-dispersibilty can be achieved by using a somewhat hydrophilic isocyanate-reactive material to prepare the product, in addition to the PNOBP. Suitable such hydrophilic isocyanate-reactive materials include polymers of ethylene oxide, random or block copolymers of ethylene oxide in which the polymerized ethylene oxide constitutes at least 50% of its weight, and compounds having one or more, preferably two or more isocyanate-reactive groups and a carboxylic acid, carboxylate, sulfonate or quaternary ammonium group, such as dimethylolpropionic acid or salts thereof. Water-dispersibility can also be imparted to the isocyanate-terminated prepolymer if the PNOBP contains a poly(ethylene oxide) block of significant length (such as about 10 or more, especially about 25 or more, oxyethylene units). Groups such as this can be introduced into the PNOBP by using a poly(ethylene oxide) polymer or copolymer as the initiator compound.

Another example of a specialized isocyanate-terminated prepolymer of the embodiments of the invention is one adapted for microcellular foam applications. In this case, a propylene oxide/ethylene oxide copolymer containing about 20 to 90% ethylene oxide by weight and having mainly primary hydroxyl groups can be used as an additional isocyanate-reactive material. Such copolymer may constitute from 10 to 60% of the weight of all isocyanate-reactive materials, and have an equivalent weight of from 1000 to about 6000. Another propylene oxide/ethylene oxide copolymer particular suitable for such an application is one having from 1 to 20% internally polymerized ethylene oxide, and an equivalent weight of from 1000 to 6000. Yet another type is a 2000-6000 equivalent weight, low unsaturation random copolymer of 85-98% by weight propylene oxide and 2-15% by weight ethylene oxide. The resulting prepolymers are particularly suitable for making molded microcellular materials such as shoe soles and frothed foam materials such as attached foam carpet cushioning. They conveniently have an isocyanate content of from 3 to 20%.

A fourth type of specialized isocyanate-functional prepolymer contains polymerizable unsaturation. One way such unsaturation can be introduced is to include a hydroxy-functional acrylate or methacrylate compound in the isocyanate-reactive materials. Suitable such hydroxy-functional acrylates and methacrylates include, for example, hydroxyalkyl esters of acrylic or methacrylic acid, and addition products of an alkylene oxide such as ethylene, propylene or butylenes oxide with acrylic or methacrylic acid. Examples of suitable hydroxy-functional acrylates and methacrylates include 2-hydroxyethyl acrylate, 2-hydroxyethyl methacrylate (HEMA), 2-hydroxylpropyl acrylate, 2-hydroxypropyl methacrylate, 4-hydroxy-n-butyl acrylate, 2-hydroxy-n-butyl acrylate, 2-hydroxy-n-butyl methacrylate, 4-hydroxy-n-butyl methacrylate, poly(oxyethylene)- and/or poly(oxypropylene)-esters of acrylic or methacrylic acid, wherein the number of oxyethylene and/or oxypropylene groups is preferably from 2 to 10, and the like. Of the foregoing, the methacrylates are preferred. HEMA is especially preferred. The presence of this unsaturation allows the prepolymer to be cured via a free-radical polymerization and/or via an ultraviolet (UV) curing mechanism. For these applications, up to 100% of the isocyanate-groups can be capped with the hydroxyl-functional acrylate or methacrylate compound. It may be desirable to form a prepolymer containing both isocyanate groups and ethylenically unsaturated groups by capping only a portion (such as from 10-95%) of the isocyanate groups. For applications in which UV curing is desired, the prepolymer can be blended with a polymerization promoter and, if desired, one or more additional polymerizable ethylenically unsaturated monomers. An example of such an application is described in U.S. Pat. No. 6,699,916. UV-curable systems containing such prepolymers can be used as adhesives and structural foams, or to make a variety of soft or hard coating materials as are used for electronics such as cell phones and military or wood-coating applications.

It is also possible to add a smaller proportion of acrylate or methacrylate groups to the prepolymer to improve adhesion to certain substrates. In such a case, it is generally suitable to use from about 0.01 to about 0.10 equivalent of hydroxyl-functional acrylate or methacrylate per isocyanate equivalent provided by the polyisocyanate. A preferred range is from about 0.02 to about 0.08 equivalent of hydroxyl-functional acrylate or methacrylate per equivalent of isocyanate compound(s). A more preferred range is from about 0.025 to about 0.06 equivalent/equivalent. The resulting acrylate- or methacrylate-functional prepolymer is useful for making hybrid urethane-acrylate or urethane-methacrylate polymers having various (i.e., IPN, core-shell or other) morphologies, and for making one- or two-part structural adhesive foams for automotive and other applications.

Isocyanate-terminated prepolymers may be blocked to form heat-activated isocyanate-functional materials.

Hydroxyl-Functional Urethanes

By using the isocyanate-reactive materials in excess, the prepolymer of the embodiments of the invention can be provided with hydroxyl functionality. In general, hydroxyl-terminated prepolymers can be prepared using the same starting materials as described above, the difference being in the relative ratios of components. As before, the selection of particular starting materials, including the use of additional isocyanate-reactive materials such as polyether polyols, polyester polyols, chain extenders and/or crosslinkers, allows a wide range of hydroxyl-terminated prepolymers to be made that are tailored for specific applications.

The hydroxyl-terminated prepolymers of the embodiments of the invention are useful in a wide variety of polyurethanes applications. They can in general be used analogously to the isocyanate-terminated prepolymers, with the exception of course that the hydroxyl-terminated materials will be cured using polyisocyanate compounds. The hydroxyl-terminated prepolymers may be used in conjunction with other isocyanate-reactive materials as needed or desirable for particular polyurethane applications.

Applications of particular interest include various water-borne and non-aqueous coating, adhesive and sealant applications. In such applications, the hydroxyl-terminated prepolymer is dispersed into an aqueous phase, and cured by mixing the dispersion with a polyisocyanate. A water-borne polyisocyanate emulsion is particularly suitable for these applications. The polyisocyanate may have isocyanurate, carbodiimide, biuret, allophonate or other linkages, which may produce a higher functionality polyisocyanate. Hard coatings made in this manner are useful coatings for military equipment, automotive equipment, automotive windshields, goggles and other eyewear and the like.

Hydroxyl-terminated prepolymers of the embodiments of the invention can also polymerize with reactive compounds other than polyisocyanates, which nonetheless contain hydroxyl-reactive groups. Examples of such reactive compounds include, for example, carboxylic acid anhydrides, particularly cyclic anhydrides, which can react with the hydroxyl-terminated prepolymer to form a polyester. Diester and dicarboxylic acid compounds can engage in transesterification reactions with the hydroxyl-terminated prepolymer, again to form polyesters. The hydroxyl-terminated prepolymer can be used as a crosslinking agent for polycarboxylic acid-containing polymers, such as polymers and copolymer of acrylic or methacrylic acid.

Prepolymers Containing Other Functional Groups

Both the isocyanate-functional prepolymers and the hydroxyl-functional prepolymers of the embodiments of the invention contain functional groups that can be used to introduce other types of functionality to the urethane. Examples of such functionality include carboxylic acid, carboxylic acid anhydride, epoxide, ethylenic unsaturation, amino, silane, and the like. This functionality can be introduced in several ways. One approach is to chemically modify the existing functional group to convert it to another desirable functional group. An example of this is to convert terminal isocyanate groups to urethane or urea groups by reaction with a monoalcohol or water, and then hydrolyze the urethane groups to form terminal primary amino groups. A second example is to oxidize terminal hydroxyl groups to form carboxylic acid groups.

Another approach is to react the existing functional groups with a bireactive material that contains both the new functional group that is desired, and a second reactive group that will react with the existing functional group on the urethane to form a covalent bond.

Bireactive materials useful for imparting specialized functionality to isocyanate-terminated urethanes include hydroxy- or amino-functional ethylenically unsaturated compounds such as hydroxyalkyl acrylates and methacrylates, aminoalkyl acrylates and methacrylates, hydroxyl-functional carboxylic acids and carboxylic acid anhydrides, hydroxyl-containing epoxide compounds such as bisphenol-A/glycidyl ether-type epoxies, hydroxyl- or amino-functional alkoxy silanes, and the like. Diamines used in excess can be used to introduce terminal amino groups to an isocyanate-terminated prepolymer.

Bireactive materials useful for imparting specialized functionality to hydroxyl-terminated urethanes include ethylenically unsaturated isocyanates such as isocyanatoethylmethacrylate, ethylenically unsaturated carboxylic acids, acid halides or acid anhydrides, epoxy-functional isocyanates, carboxylic acids, acid halides or acid anhydrides, ethylenically unsaturated alkoxy silanes such as vinyl trimethoxysilane, as well as many others.

Methods of making epoxy-functional adducts from hydroxy-functional materials are described, for example, in U.S. Pat. No. 4,599,401 and EP 139,042, EP 143,120 and EP 142,121—those methods can be adapted to the use of the urethanes described herein. A particular method of introducing terminal alkoxysilane groups is described in U.S. Pat. No. 6,762,270.

Prepolymers of the embodiments of the invention that have ethylenic unsaturation are useful as UV curable materials and as raw materials in polyurethane adhesives, sealants and/or structural foam applications, as described before.

Prepolymers of the embodiments of the invention having carboxylic acid or carboxylic anhydride functional groups are useful for making polyesters, and a crosslinking agents for polymers having pendant hydroxyl groups, such as polyvinyl alcohol or polymers of a hydroxyalkyl acrylate or methacrylate, for example.

Prepolymers of the embodiments of the invention that have silane functionality, in particular hydrolyzable silane groups such as mono-, di- or trialkoxysilane groups, are useful in a wide range of sealant, elastomer, coating and adhesive applications in which moisture curing is desired.

Epoxy-functional prepolymers of the embodiments of the invention can be cured using well-known amine curing agents to form epoxy resins for adhesive, electronics potting, and other applications.

As discussed, the prepolymer of the embodiments of the invention is useful in a wide variety of polyurethane-forming reactions, as well as an intermediate in making other types of polymers such as polyesters, polycarbonates, vinyl-polyurethane hybrid polymers, and the like. The types of polymer for which the prepolymer is useful will of course be determined largely by the particular functional groups that are present.

In general, polyurethanes can be prepared by reacting an isocyanate-functional prepolymer of the embodiments of the invention with water and/or additional isocyanate-reactive materials. Methods for accomplishing this are well known in the art. General methods for making cast and microcellular elastomers are described, for example, in U.S. Pat. Nos. 5,648,447, 6,022,903, 5,856,372 and EP 868,455, among many others. Methods for making polyurethane sealant and adhesive compositions are described in U.S. Pat. Nos. 4,985,491, 5,774,123, 6,103,849, 6,046,270 and 6,512,033, among others. Aqueous polyurethane dispersion production methods are described, for example, in U.S. Pat. Nos. 4,792,574, 6,444,746 and 6,455,632. Reaction injection molding methods for making polyurethane and/or polyurea polymers are described, for example, in U.S. Pat. Nos. 4,876,019, 4,766,172, 4,433,067 and 4,218,543. General methods for making rebond foam are described in U.S. Pat. No. 5,817,703.

The prepolymer can be blended with a variety of types of useful additives, including, for example, surfactants, catalysts, pigments, dyes, fillers, dryers, rheological and viscosity modifiers, dispersants, surfactants, preservatives, antimicrobials, pesticides, fertilizers and the like.

### EXAMPLES

The following examples are provided to illustrate the embodiments of the invention, but are not intended to limit the scope thereof. All parts and percentages are by weight unless otherwise indicated.

The following materials are used:
VORANOL* 220-056N A propylene glycol initiated polyoxypropylene based diol with molecular weight of about 2000 and a hydroxyl equivalent weight of about 1000. Available from The Dow Chemical Company.
VORANOL* CP-6001 A glycerin initiated 2000 equivalent weight propoxylated triol capped with 15% Ethylene oxide. Available from The Dow Chemical Company.
PNOBP-D A 2-functional polyether natural oil based polyol prepared using fatty acids from soy oil. It is made by reacting hydroxymethylated soybean fatty acid methyl ester monomers with a 1000 equivalent weight poly(propylene/ethylene oxide) diol initiator. The diol initiator is made by taking propylene glycol and alkoxylating it with propylene oxide until a molecular weight of 1750 is reached followed by alkoxylation using ethylene oxide until a molecular weight of 2000 is reached. The monomer to diol initiator molar ratio is 3.5. PNOBP-D has an equivalent molecular weight of about 1390, a hydroxyl number (OH#) of 40, and a renewable content of 37%, calculated as the renewable carbon in the polyol (carbon from plant sources) as a fraction of the total carbon in the polyol.
PNOBP-T A 3-functional polyether natural oil based polyol prepared using fatty acids from soy oil. It is made by reacting hydroxymethylated soybean fatty acid methyl ester monomers with a 1500 equivalent molecular weight poly(propylene/ethylene oxide) triol initiator. The triol initiator is made by taking glycerin and alkoxylating it with propylene oxide until a molecular weight of 3675 is reached followed by alkoxylation using ethylene oxide until a molecular weight of 4500 is reached. The monomer to triol initiator molar ratio is 6. PNOBP-T has an equivalent molecular weight of about 2000, a hydroxyl number (OH#) of 28, and a renewable content of 32%.
ISONATE* OP-50 A 50 percent 4,4′-methylene diphenyl isocyanate, 50 percent 2,4′-methylene diphenyl isocyanate mixture. Available from The Dow Chemical Company.
ISONATE* OP-25 A 75 percent 4,4′-methylene diphenyl isocyanate, 25 percent 2,4′-methylene diphenyl isocyanate mixture. Available from The Dow Chemical Company.
ISONATE* 143 L A polycarbodiimide-modified diphenylmethane diisocyanate. Available from The Dow Chemical Company.
PAPI* 94 A polymeric methylene diisocyanate containing approximately 98% of 4,4′ isomer with the remaining 2% being the 2,4′ isomer, and having a functionality of 2.3. Available from The Dow Chemical Company.
VORANATE* T-80 A toluene diisocyanate (80% 2,4-toluene diisocyanate and 20% 2,6-toluene diisocyanate by weight) composition available from The Dow Chemical Company.
DMDEE 2,2′-Dimorpholino diethyl ether, 97% purity, used as received. Available from Sigma-Aldrich Company.
   *ISONATE, PAPI, VORANATE, and VORANOL, are trademarks of The Dow Chemical Company.

The prepolymers are synthesized in 1 L glass reactors equipped with a thermometer, overhead stirrer, nitrogen inlet and outlet for continuous flow of nitrogen, and chemicals addition inlet. The prepolymers are prepared according to a standard prepolymer procedure method:
1. The isocyanate blends are prepared and the acidity may optionally be regulated by adding benzoyl chloride until a targeted acidity value is reached.
2. The isocyanate premix is loaded into the glass reactor and heated to 60° C.
3. The polyols are premixed and added as a blend into the reaction vessel at a controlled rate, while maintaining the reaction mixture at about 60° C. to about 70° C.
4. At the end of the polyol addition the temperature is heated at 70° C. for 3 hours.
5. Samples are taken periodically to determine the NCO content.

All characterizations of the prepolymers are performed three days after the synthesis. The prepolymers are characterized in terms of their percent NCO content in the final prepolymer. The isocyanate content (wt % NCO) is determined by a titration technique according to the ASTM methods D5155-96, Test Method C.

Viscosity is measured using a TA instrument ARES Rheometer. An MP 303 parallel plate fixture is employed in conjunction with a Peltier cooled base plate configuration. The upper plate is 40.0 mm in diameter and the distance between the two plates is maintained at 0.50 mm. All measurements are performed in steady shear mode at a shear rate of 10 s⁻¹. measurements are performed over the temperature range of 20° C. to 60° C. employing a heating rate of 10° C. per minute. The samples are loaded at 20° C.

Reactivity characteristics of the prepolymer are measured to determine curing time and open time (also called handling time). In some applications a short curing time may be desired while at the same time maintaining a long enough open time for handling and mixing. The reactivity is generated using the following Dow methods:

Method 1. Intrinsic reactivity of the prepolymer with 0.4 weight % DMDEE catalyst is measured on a 200 μm thick film of the prepolymer using a Byk drying recorder. Open time and cure time is measured under room temperature and at about 50% relative humidity.

Method 2. Tack free time is measured on a prepolymer/water mixture (90/10 weight %) foamed at room temperature and at 100° C. DMDEE (0.4 weight %) is added to the prepolymer to enhance reactivity. Cream time is the time it takes for the prepolymer/water reaction to result in foaming, string time is the time it takes for the prepolymer to become tacky, and the tack free time is the time it takes for the prepolymer to be fully cured and tack free.

Method 3. Foaming profile at 20° C. of a prepolymer/water mixture (90/10 weight %) versus time with a Foamat Qualification System.

Moisture cured polyurethane films are prepared by drawing the prepolymer on a TEFLON sheet dusted with talcum powder. The films are moisture cured at room temperature and ambient humidity (typically at 23° C. and 50% relative humidity) for one week before analysis.

Tensile Strength, % Elongation, and Stress @ 100 psi (modulus) is measured according to ASTM D1708.

Water uptake of the cured polyurethane films are measured a cutting piece of the polyurethane film and weighing it to obtain weight W₀. The film piece is then immersed in de-ionized water for 24 hours. The film piece is then wiped with a tissue and weighed to obtain W₁. The water absorption is calculated using the equation:

Water uptake=(W₁−W₀)/W₀*100%

### Comparative Example 1 and Examples 1-4

Comparative Example 1 (CE1) and Examples 1-4 (E1-4) are made and tested as described above. The composition and experimental data are given in Table 1:

TABLE 1

| | CE1 | El | E2 | E3 | E4 |
|---|---|---|---|---|---|
| VORANOL* 220-056N | 30 | 30 | 30 | | 15 |
| VORANOL* CP-6001 | 33 | | 16.5 | | |
| PNOBP-D | | | | 30 | 15 |
| PNOBP-T | | 33 | 16.5 | 33 | 33 |
| ISONATE* OP-50 | 31 | 31 | 31 | 31 | 31 |
| PAPI 94 | 6 | 6 | 6 | 6 | 6 |
| Prepolymer Renewable Content | Less | 10.6 | 5.5 | 21.5 | 16.1 |
| | than 1 | | | | |
| EO content, % | 4.62 | 6.6 | 5.53 | 10.2 | 8.4 |
| Theoretical Prepolymer % NCO | 10.4 | 10.3 | 10.3 | 10.8 | 10.6 |
| content | | | | | |
| Measured Prepolymer % NCO | 10.4 | 10.52 | 10.4 | 10.7 | 10.53 |
| content | | | | | |
| Open time, hours | 0.5 | 0.5 | 0.5 | 0.5 | 0.5 |
| Cure time, hours | 2 | ~3 | 2 | 5.5 | 4.5 |
| Cream time 25° C., minutes | 0.1 | | | 0.8 | |
| Cream time 100° C., minutes | | | | 0.6 | |
| String time 25° C., minutes | 7.3 | | | 13.46 | |
| String time 100° C., minutes | 3.3 | | | 4.1 | |
| Tack free time 25° C., minutes | 15 | | | 24.4 | |
| Tack free time 100° C., minutes | 5.4 | | | 5.5 | |
| Water Uptake, % | 1.68 | 1 | 0.67 | 1 | 0.8 |
| Elongation lost, % | 20 | 9 | 5 | 11 | 12 |

Targeted theoretical % NCO content for Examples E1-E4 is kept close enough to that of the Comparative Example CE1 to prevent disparity in results that could be contributed to difference in hard segment content of the final polymer.

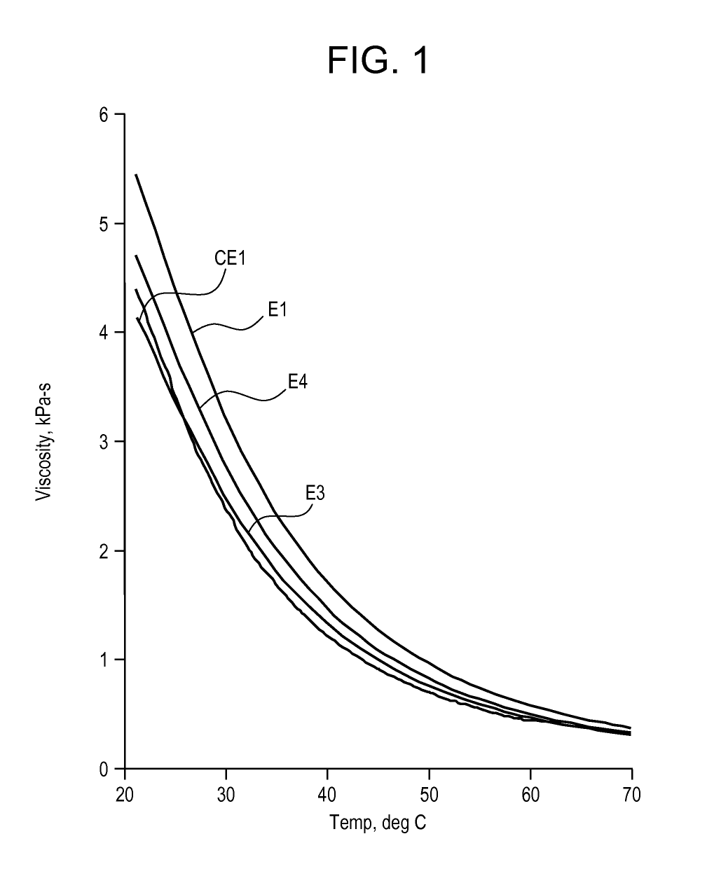

FIG. 1 is a plot of viscosity versus temperature for CE1, E1, E3, and E4. The viscosity of the PNOBP prepolymers (E1, E3, and E4) are within a similar range as the non-PNOBP prepolymer (CE1).

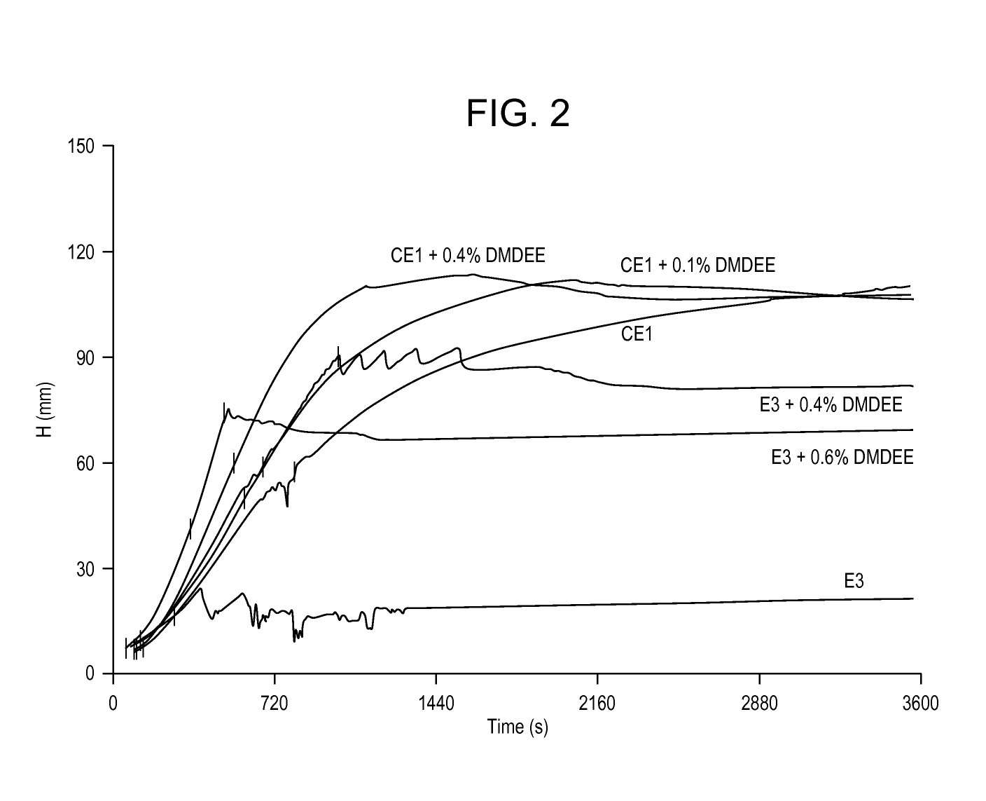

FIG. 2 is a plot of the foaming profiles (height versus time) of the PNOBP prepolymer having the highest renewable content (E3) and the non-PNOBP prepolymer (CE1). Foaming profiles are collected both with and without DMDEE catalyst.

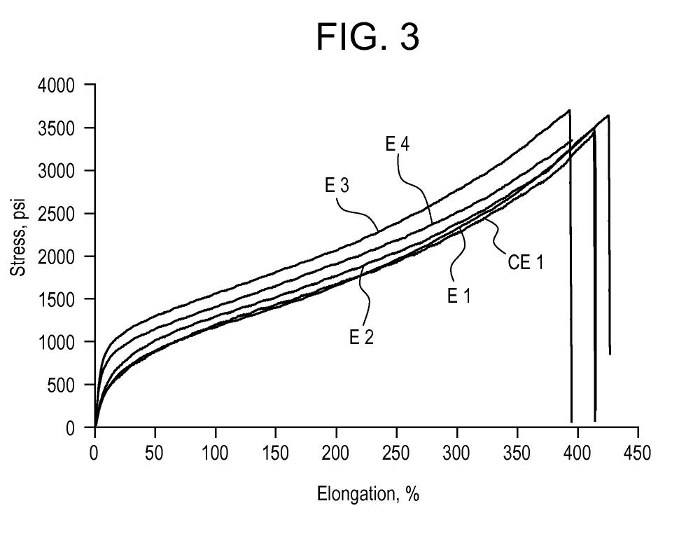

FIG. 3 is a plot of stress-strain behavior of the resulting polyurethane films. The results for the films of the PNOBP prepolymers (E1-E4) are similar to the results for the film of the non-PNOBP prepolymer (CE1).

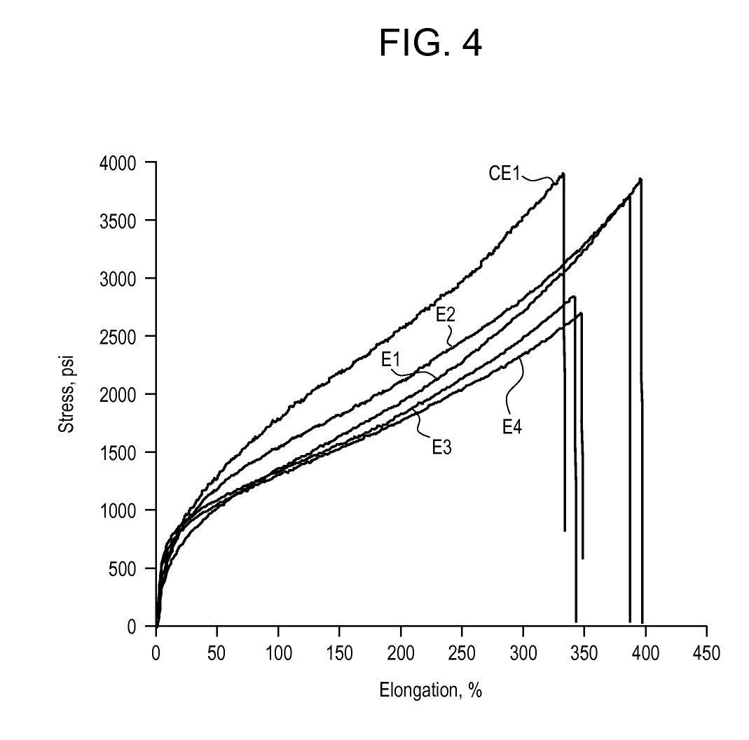

FIG. 4 is a plot of stress-strain behavior of resulting polyurethane films after water exposure. As also enumerated in Table 1, the film made from the non-PNOBP prepolymer had the greatest % loss of elongation of the film after 24 hours of exposure to water. The films made from PNOBP prepolymer have significantly less % loss of elongation. Even though the polymer films made using the PNOBP prepolymers have more E0 content than the films made using the non-PNOBP prepolymer, the films made using the PNOBP prepolymers have less water uptake than the film made using non-PNOBP prepolymer. This may be a result of the hydrophobicity of the PNOBPs which result in less loss of elongation.

While the foregoing is directed to embodiments of the present invention, other and further embodiments of the invention may be devised without departing from the basic scope thereof, and the scope thereof is determined by the claims that follow.

1. A prepolymer having at least one urethane group, comprising the reaction product of at least one isocyanate and at least one natural oil based polyol, wherein the natural oil based polyol comprises at least two natural oil moieties separated by a molecular structure having an average of at least about 19 ether groups between any 2 of the natural oil moieties or by a polyether molecular structure having an equivalent weight of at least about 480.

2. A method for producing a prepolymer having at least one urethane group, the method comprising reacting, at least:
at least one isocyanate with
at least one natural oil based polyol to form the prepolymer, wherein the at least one natural oil based polyol comprises at least two natural oil moieties separated by a molecular structure having an average of at least about 19 ether groups between any 2 of the natural oil moieties or by a polyether molecular structure having an equivalent weight of at least about 480.

3. A polyurethane product comprising the reaction product of at least one isocyanate-reactive material and at least one prepolymer, wherein the prepolymer is isocyanate-functional and comprises the reaction product of at least one isocyanate and at least one natural oil based polyol, wherein the natural oil based polyol comprises at least two natural oil moieties separated by a molecular structure having an average of at least about 19 ether groups between any 2 of the natural oil moieties or by a polyether molecular structure having an equivalent weight of at least about 480.

4. A method for producing a polyurethane product, the method comprising reacting, at least:
at least one isocyanate-reactive material with
at least one prepolymer, wherein the prepolymer is isocyanate-functional and comprises the reaction product of at least one isocyanate and at least one natural oil based polyol, wherein the natural oil based polyol comprises at least two natural oil moieties separated by a molecular structure having an average of at least about 19 ether groups between any 2 of the natural oil moieties or by a polyether molecular structure having an equivalent weight of at least about 480.

5. The prepolymer, polyurethane product claim 1, wherein the natural oil based polyol is produced using a natural oil or derivative thereof to react with a polyether initiator.

6. The prepolymer of claim 5, wherein the polyether initiator has an average equivalent weight of at least about 600 and at most about 3000 per active hydrogen group.

7. The prepolymer of claim 5, wherein the polyether initiator has an active hydrogen functionality of at least about 2 and at most about 8.

8. The prepolymer of claim 5, wherein the polyether initiator is depicted by
R((OCH₂CHY)_{b}—XH)ₚ, Formula 1:
where Y is a HCH₃ or higher alkyl group or mixture thereof; X is an active hydrogen group; p is 1 to 8; b is sufficient to result in an equivalent weight per active hydrogen group of at least about 480; the number of ether units in an arm of the polyether initiator, b, is preferably at least about 9.5; R represents a linear, cyclic chain or combination thereof of alkane (C—C), alkene (C═C), ether (C—O—C) linkage or combination thereof; R has from 1 to 30 carbon atoms.

9. The prepolymer of claim 5, wherein each polyether NOP has an average of at least about 0.5 to at most about 8 fatty acid moieties oligomerized on each active hydrogen atom of the polyether initiator.

10. The prepolymer of claim 1, wherein the at least two natural oil moieties comprises at least one natural oil based monomer resulting from hydroformylation at sites of unsaturation on fatty acids or fatty acid methyl esters derived from at least one natural oil.

11. The prepolymer of claim 1, wherein the prepolymer has an isocyanate functionality of from 1.8 to about 4.

12. The prepolymer of claim 1, wherein the prepolymer has an isocyanate equivalent weight of from about 500 to about 50,000.

13. The prepolymer of claim 1, wherein the prepolymer further comprises at least one of amino groups, epoxide groups, and silyl groups.

14-15. (canceled)

16. The prepolymer of claim 13, wherein the silyl groups are hydrolyzable.

17. The prepolymer of claim 1, wherein the prepolymer is moisture-curable.

18. The prepolymer of claim 1, wherein the prepolymer is water-dispersible.

19. The prepolymer of claim 18, wherein prepolymer further comprises at least one carboxylic acid, carboxylic acid salt, sulfonate or quaternary ammonium group.

20. A polymer prepared by curing a prepolymer of claim 1.

21. The polymer of claim 20 which is cellular.

22. The polymer of claim 20 which is an adhesive, binder, elastomer, sealant or coating.

23. The polymer of claim 20, which is cured by reaction with moisture.

24. The polymer of claim 20, which is cured by reaction with a polyol, polyamine or aminoalcohol.
